(19) United States
(12) Patent Application Publication
(10) Pub. No.: US 2022/0402968 A1
(43) Pub. Date: Dec. 22, 2022

(54) INTENSIFIED VIRUS FILTRATION USING DIAFILTRATION BUFFER

(71) Applicant: EMD Millipore Corporation, Burlington, MA (US)

(72) Inventors: Herbert Lutz, Sudbury, MA (US); Kenneth Scott, Hooksett, NH (US); Michael Bruce, Merrimack, NH (US); Kristina Cunningham, Lexington, MA (US)

(21) Appl. No.: 17/756,096

(22) PCT Filed: Dec. 11, 2020
(86) PCT No.: PCT/US2020/064466

§ 371 Date: May 17, 2022

Related U.S. Application Data
(60) Provisional application No. 62/947,082, filed on Dec. 12, 2019.
(60) Provisional application No. 63/049,293, filed on Jul. 8, 2020.

(51) Int. Cl. C07K 1/36; C12M 1/00; C12N 7/00; C07K 1/22; C07K 1/34; B01D 15/38; B01D 15/18; B01D 15/36; B01D 15/32

(52) U.S. Cl. CPC C07K 1/36; C12M 47/12; C12N 7/00; C07K 1/22; C07K 1/34; B01D 15/3809; B01D 15/1871; B01D 15/363; B01D 15/362; B01D 15/327

(57) ABSTRACT

Method and system for purifying a sample comprising a biomolecule of interest and impurities, comprising expressing said biomolecule of interest in a bioreactor to form a product sample comprising said biomolecule of interest and impurities; subjecting said product sample to filtration to form a clarified product sample; subjecting said clarified product sample to affinity chromatography to remove impurities; subsequently subjecting said product sample to diafiltration followed by virus filtration and optional concentration. The buffer used during the diafiltration step (and thus in the virus filtration step) is the buffer desired for the final formulation of the product.

### CROSS-REFERENCE TO RELATED APPLICATIONS

This application claims the benefit of priority of U.S. Provisional Patent Application Nos. 62/947,082 filed on Dec. 12, 2019 and 63/049,293 filed on Jul. 8, 2020. The entirety of each application is incorporated herein by reference.

### FIELD

The present disclosure relates to efficient processes and systems for the purification of biological molecules including therapeutic antibodies and Fc-containing proteins.

### BACKGROUND

The general process for the manufacture of biomolecules, such as proteins or viruses, particularly recombinant proteins, typically involves two main steps: (1) the expression of the protein in a host cell, followed by (2) the purification of the protein. The first step involves growing the desired host cell in a bioreactor to induce the expression of the protein. Some examples of cell lines used for this purpose include Chinese hamster ovary (CHO) cells, myeloma (NSO) bacterial cells such as E-coli and insect cells. Once the protein is expressed at the desired levels, the protein is removed from the host cell and harvested. Suspended particulates, such as cells, cell fragments, lipids and other insoluble matter are typically removed from the protein-containing fluid in a downstream purification process, resulting in a clarified fluid containing the protein of interest in solution as well as other soluble impurities.

The second step involves the purification of the harvested protein to remove impurities that are inherent to the process. The main goal of the harvest and downstream operations is to isolate the product (e.g., expressed protein) from the soluble/insoluble impurities. Examples of impurities include host cell proteins (HCP, proteins other than the desired or targeted protein), nucleic acids, endotoxins, viruses, protein variants, protein aggregates, and cell culture media components/additives. This purification typically involves several chromatography steps, which can include one or more of affinity chromatography, cation-exchange chromatography in bind/elute mode, anion-exchange chromatography in flow through mode, hydrophobic interaction, etc. on solid matrices such as porous agarose, polymeric or glass or by membrane based adsorbers.

One example of a process template includes primary clarification by centrifugation, secondary clarification by filtration, and a chromatography process train involving protein-A affinity in bind/elute mode, followed by cation exchange in bind/elute mode, followed by anion exchange in flow through mode. The protein-A column captures the protein of interest or target protein by an affinity mechanism while the bulk of the impurities pass through the column to be discarded. The protein then is recovered by elution from the column. Since most of the proteins of interest have isoelectric points (PI) in the basic range (8-9) and therefore being positively charged under normal processing conditions (pH below the PI of the protein), they are bound to the cation exchange resin in the second column. Other positively charged impurities are also bound to this resin. The protein of interest is then recovered by elution from this column under conditions (pH, salt concentration) in which the protein elutes while the impurities remain bound to the resin. The anion exchange column is typically operated in a flow through mode, such that any negatively charged impurities are bound to the resin while the positively charged protein of interest is recovered in the flow through stream. Following the downstream purification process, virus filtration can be carried out, followed by ultrafiltration/diafiltration to condition the buffer system and to concentrate the product prior to the final fill unit operation to complete the manufacturing process. This process results in a highly purified and concentrated protein solution, which can be critical particularly where the therapeutic proteins are meant for use in humans and have to be approved by health authorities, such as the Food and Drug Administration (FDA).

FIG. 1 illustrates one conventional process. The process includes a cell harvest step, which may involve use of centrifugation to remove cell and cell debris from a cell culture broth, followed by depth filtration. The cell harvest step is usually followed by a capture step such as a Protein A affinity purification step, which is followed by virus inactivation. Virus inactivation is typically followed by one or more chromatographic steps, also referred to as polishing steps, which usually include one or more of cation exchange chromatography and/or anion exchange chromatography and/or hydrophobic interaction chromatography and/or mixed mode chromatography and/or hydroxyapatite chromatography. The polishing steps are followed by virus filtration and ultrafiltration/diafiltration, which completes the process.

The capture step may use Protein-A resin such as Eshmuno® A resin, a rigid, Protein A affinity chromatography resin or ProSep® Ultra Plus media, both commercially available from EMD Millipore Corporation, especially for antibodies containing Fc regions. Other resins operated in bind and elute mode also may be suitable for capture. Bind and elute chromatography includes (1) product loading to a target binding capacity, (2) elution of product from the column, and (3) cleaning to prepare the resin for re-use.

One issue during virus filtration is increases in filter resistance as psi/LMH (or loss in permeability as LMH/psi, where LMH is liters/m2/h) during the course of filtering the feed stream at a filter throughput as L/m2. Put in these terms, one can compare pressure rises during constant feed flow operation with flow decays during constant pressure filtration. These changes are commonly described as filter plugging and can significantly limit the L/m2 filter throughput and thus increase the processing cost as S/L. Some mechanisms that can cause plugging include retention of high molecular weight (HMW) impurities or retention of aggregates of the therapeutic protein product. As the aggregates increase in size and approach the size of the virus that the membranes used for virus filtration are meant to retain, they can deposit on the surface of the membranes and in the pores. This deposition can be related to the permeability loss. Proteins can also be partially retained by a filter, increasing their internal concentrations, viscosity, and osmotic pressure within the filter to create a plugging effect.

Size based membrane filtration provides critical virus removal assurance in most biopharmaceutical molecule production processes. Health authorities expect that every therapeutic protein produced in mammalian viruses uses a virus filtration step to manage the risk of virus contamination in order to grant marketing approval. Many studies have examined the mechanisms behind the formation of product aggregates, how these aggregates foul virus removal filters, and how to maintain filter flux by removing aggregates with adsorptive prefilters. After some throughput, prefilters may become saturated with aggregates or HMW, and these species start breaking through and begin to plug the filter. New modalities (e.g. bispecific antibodies) in concert with the higher product concentrations associated with intensified processing have made occurrences of premature filter plugging more common, even with implementation of adsorptive prefilters.

Biotherapeutic formulation involves finding diafiltrate buffer solution conditions that promote stability and avoid aggregation in the vial for a long shelf life. Using small volume analytical techniques that relate protein properties to aggregation, biotherapeutic formulation chemists rapidly screen multiple buffer systems and excipient concentrations to develop an appropriate diafiltrate solution. These objectives mirror those of finding virus filter feed solution conditions to avoid virus filter plugging. Accordingly, one can consider moving the location of the virus filtration step from before (i.e., upstream of) UF/DF to after (i.e., downstream of) UF/DF to take advantage of DF buffer stabilizing effects and plugging reduction. This could enable one to run higher throughputs or at higher concentrations than previously so as to manufacture biomolecules such as monoclonal antibodies (Mabs) in a more efficient and cost-effective manner.

### SUMMARY

Problems of the prior art have been overcome by the embodiments disclosed herein, which provide a method and system for the manufacture of biomolecules. In certain embodiments, virus filtration is carried out with the biomolecule in a diafiltrate buffer. Preferably the diafiltrate buffer is the same buffer used in the final formulation of the product to ensure product stability and long shelf-life. Reduced virus filtration area can be achieved.

Embodiments disclosed herein include purification and isolation of biomolecules of interest derived from cell culture fluids. In certain embodiments, methods and systems disclosed include concentration followed by a downstream purification process. In certain embodiments, the downstream purification process may include sequential purification by one or more chromatography columns.

In certain embodiments, a method for purifying a sample comprising a biomolecule of interest and impurities is disclosed, the method comprising expressing the biomolecule of interest in a bioreactor to form a product sample comprising the biomolecule of interest and impurities; subjecting the product sample to filtration to clarify the product sample; and subjecting the concentrated product sample to affinity chromatography to remove impurities from it. In certain embodiments, the product sample (e.g., harvested cell culture) from the bioreactor is subjected to one or more clarification steps prior to being subjected to affinity chromatography. The one or more clarification steps may include one or more of centrifugation, tangential flow filtration, depth filtration, and sterile filtration.

In certain embodiments, the affinity chromatography uses Protein A affinity ligand.

In certain embodiments, the method further comprises subjecting the concentrated product sample to a virus inactivation step.

In certain embodiments, the method comprises subjecting the concentrated product sample to a polishing step. In some embodiments, the polishing step comprises one or more of anion exchange chromatography, cation exchange chromatography, and hydrophobic interaction chromatography. In some embodiments, both virus inactivation and polishing may be carried out.

In certain embodiments, a diafiltration step is carried out prior to virus filtration. In some embodiments, the diafiltration step is carried out directly upstream of virus filtration with no unit operations in between. In various embodiments, the buffer or buffers used in the diafiltration step are chosen to match the buffer or buffers desired for the final formulation of the product. As a result, the buffer used during the subsequent virus filtration step is the same buffer desired for the final formulation.

In certain embodiments, the biomolecule is an antibody selected from the group consisting of a recombinant antibody, a recombinant monoclonal antibody, a polyclonal antibody, a humanized antibody and an antibody fragment. In some embodiments, the biomolecule is a protein. In other embodiments, the biomolecule is a virus such as an Associated Adenovirus (AAV) vector used for gene therapy. In such a case one may be using virus filtration to pass AAV product while retaining larger adventitious or endogenous virus contaminants.

In certain embodiments, a system for purifying a biomolecule of interest is disclosed, the system comprising a bioreactor; a filter such as a depth filter downstream of the bioreactor for clarification; at least two affinity chromatography columns configured in series downstream of the filter; a virus inactivation step or a virus filter removal step downstream of the at least two affinity chromatography columns; one or more anion exchange, cation exchange or hydrophobic interaction exchange chromatography columns positioned downstream of the virus inactivation step or virus filter removal step for purification/polishing; one or more ultrafilters configured to run in a concentration mode or in a diafiltration mode positioned downstream of the purification/polishing unit or units (e.g., for batch TFF operation, this can be the same filter run sequentially in different ways, and for single pass TFF, this can be sequential filters run in different ways); a virus filter downstream of the one or more ultrafilter; and optionally an ultrafiltration unit for concentrating the biomolecule of interest downstream of the virus filter.

In some embodiments, the at least two affinity chromatography columns each comprise Protein A affinity ligand. In some embodiments, there are exactly two affinity chromatography columns.

### BRIEF DESCRIPTION OF THE DRAWINGS

FIG. 1 is a schematic representation of a conventional purification process used in the industry; and

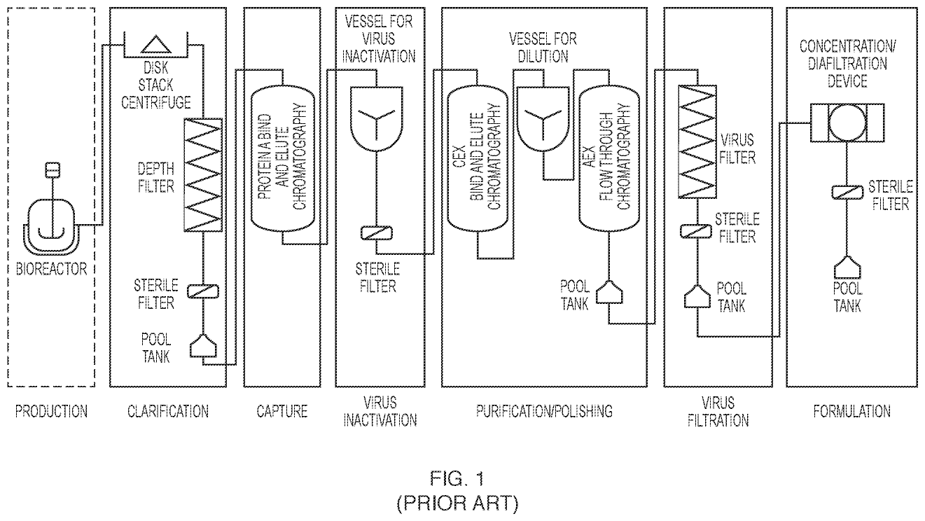

FIG. 2 is a schematic representation of a purification system in accordance with certain embodiments.

### DETAILED DESCRIPTION

In the following description, the terms “selected biomolecule”, “target biomolecule” or “molecule”, “target protein”, “biomolecule or protein of interest”, or similar terms all refer to products of a biomolecule manufacturing process.

The terms “contaminant,” “impurity,” and “debris,” as may be used interchangeably herein, refer to any foreign or objectionable molecule, including a biological macromolecule such as a DNA, an RNA, one or more host cell proteins, endotoxins, lipids, protein aggregates and one or more additives which may be present in a sample containing the product of interest that is being separated from one or more of the foreign or objectionable molecules. Additionally, such a contaminant may include any reagent which is used in a step which may occur prior to the separation process.

As used herein, the term “sample” refers to any composition or mixture that contains a target molecule, such as a target protein, to be purified. Samples may be derived from biological or other sources. Biological sources include eukaryotic and prokaryotic sources, such as plant and animal cells, tissues and organs. In some embodiments, a sample includes a biopharmaceutical preparation containing a protein of interest to be purified. In a particular embodiment, the sample is a cell culture feed containing a protein of interest to be purified. The sample may also include diluents, buffers, detergents, and contaminating species, debris and the like that are found mixed with the target protein or protein of interest. The sample may be “partially purified” (i.e., having been subjected to one or more purification steps, such as filtration steps) or may be obtained directly from a host cell or organism producing the target molecule (e.g., the sample may comprise harvested cell culture fluid).

As used herein, the phrase “consisting essentially of” limits the scope of a claim to the specified materials or steps and those that do not materially affect the basic and novel characteristics of the claimed subject matter. The term permits the inclusion of elements or steps which do not materially affect the basic and novel characteristics of the apparatus, system or method under consideration. Accordingly, the expressions “consists essentially of” or “consisting essentially of” mean that the recited embodiment, feature, component, step, etc. must be present and that other embodiments, features, components, steps, etc., may be present provided the presence thereof does not materially affect the performance, character or effect of the recited embodiment, feature, component, step, etc. The presence of an operation or step that has no material effect on the sample or product is permitted. For example, a method consisting essentially of purifying a sample comprising a biomolecule of interest and impurities, consisting essentially of expressing said biomolecule of interest in a bioreactor to form a product sample comprising said biomolecule of interest and impurities; subjecting said product sample to filtration to form a clarified product sample; subjecting said concentrated product sample to affinity chromatography to remove impurities from said concentrated product sample; subjecting said product sample to virus inactivation; subjecting said product sample to purification/polishing such as with ion exchange chromatography; subjecting said product sample to buffer exchange by diafiltration, followed by subjecting said sample to virus filtration, excludes other steps or unit operations carried out between the bioreactor and the virus filtration operations, particularly between the diafiltration and virus filtration, that would materially change the composition of the product sample.

In certain embodiments, the sample that is the starting material of the process may vary depending upon the cell line in which it was grown as well as the conditions under which it is grown and harvested. For example, in most CHO cell processes the cells express the molecule outside of the cell wall into the media. One tries not to rupture the cells during harvest in order to reduce the amount impurities in the mixture. However, some cells during growth and harvesting may rupture due to shear or other handling conditions or die and lyse, spilling their contents into the mixture. In bacteria cell systems, the biomolecule is often kept with the cellular wall or it may actually be part of the cellular wall (Protein A). In these systems the cell walls need to be disrupted or lysed in order to recover the biomolecule of interest.

The target molecule to be purified can be any biomolecule, preferably a protein, in particular, recombinant protein produced in any host cell, including but not limited to, Chinese hamster ovary (CHO) cells, Per.C6® cell lines available from Crucell of the Netherlands, myeloma cells such as NSO cells, other animal cells such as mouse cells, insect cells, or microbial cells such as E. coli or yeast. Additionally, the mixture may be a fluid derived from an animal modified to produce a transgenic fluid such as milk or blood that contains the biomolecule of interest. Optimal target proteins are antibodies, immunoadhesins and other antibody-like molecules, such as fusion proteins including a C_{H}2/C_{H}3 region. For example, this product and process can be used for purification of recombinant humanized monoclonal antibodies such as (RhuMAb) from a conditioned harvested cell culture fluid (HCCF) grown in Chinese hamster ovary (CHO) cells expressing RhuMAb.

In certain embodiments, in the downstream purification process, a series of purification media having the desired chemical functionalities, are used to effect the removal of soluble impurities while the product remains in solution and flows through the purification media, resulting in a purified stream containing the product. Suitable forms of purification media include derivatized membranes, functionalized chromatography media, or any other porous material having the desired chemical functionality to interact with the various impurities so that the media can capture the impurities by electrostatic, hydrophobic, or affinity interactions. In view of the complex and varied natured of the impurities, a multitude of purification media having different chemical functionalities can be arranged in series to remove a variety of impurities having different chemical properties.

In certain embodiments, disclosed is a process for purifying a target molecule from a sample, where the process comprises: (a) expressing a protein in a bioreactor to form a protein sample; (b) subjecting the protein sample to filtration such as depth filtration; (c) subjecting the resulting protein sample to Protein A affinity chromatography, which employs one or more affinity chromatography units.

Also disclosed is a system for purifying a target molecule from a sample, comprising a bioreactor; a filter unit such as a depth filtration unit; one or more affinity chromatography columns such as one or more Protein A affinity chromatography columns in fluid communication with the filtration unit; optionally one or more virus inactivation units downstream of the one or more affinity chromatography columns; optionally a polishing phase downstream of the affinity chromatography columns, which may include one or more of an anion exchange chromatography column, a cation exchange chromatography column, and a hydrophobic interaction chromatography column; a diafiltration unit; a virus filtration unit; and an ultrafiltration unit downstream of the virus filtration unit.

In some embodiments, there is a connecting line between the various devices in the system. The devices are connected in line such that each device in the system is in fluid communication with devices that precede and follow the device in the system.

In some embodiments, the bioreactor used in a system according to the present invention is a disposable or a single use bioreactor. In some embodiments, the system is enclosed in a sterile environment.

In some embodiments, the starting sample is a cell culture. Such a sample may be provided in a bioreactor. In certain embodiments, the bioreactor is a perfusion bioreactor.

In some embodiments, the capture step may include bind and elute chromatography apparatus that includes at least two separation units, with each unit comprising the same chromatography media, e.g., Protein A affinity media. In a particular embodiment, the Protein A media comprises a Protein A ligand coupled to a rigid hydrophilic polyvinylether polymer matrix. In other embodiments, the Protein A ligand may be coupled to agarose or controlled pore glass. The Protein A ligand may be based on a naturally occurring domain of Protein A from Staphylococcus aureus or be a variant or a fragment of a naturally occurring domain. In a particular embodiment, the Protein A ligand is derived from the C domain of Staphylococcus aureus Protein A. The separation units are connected to be in fluid communication with each other in series, such that a liquid can flow from one separation unit to the next.

Diafiltration may be used for buffer exchange, desalting and/or sample concentration, such as to remove, replace, or lower the concentration of salts or solvents from solutions containing biomolecules of interest. In certain embodiments, sample may be circulated over an ultrafiltration membrane and returned to the retentate vessel, where fresh buffer is added, while permeate removed.

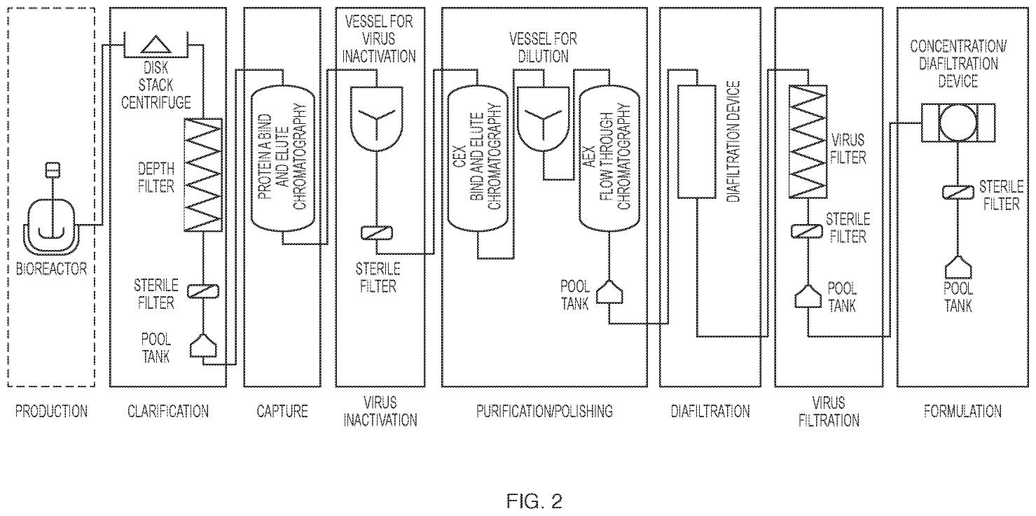

In some embodiments, as seen in FIG. 2, upstream of virus filtration, preferably directly upstream thereof, the sample containing the target molecule is subjected to diafiltration, which typically employs the use of an ultrafiltration membrane in a Tangential Flow Filtration (TFF) mode. In case of Tangential Flow Filtration (TFF), the fluid is pumped tangentially along the surface of the filter media and applied pressure serves to force a portion of the fluid through the filter medium to the filtrate side. Diafiltration results in the replacement of the fluid which contains the target molecule with the desired buffer, and allows for proper conditioning or adjustment of the solution conditions, including pH and conductivity. An ultrafilter may be used either in a concentration mode or in a diafiltration model. For batch TFF operation, these can be the same filters run sequentially in different ways. For single pass TFF, these can be sequential filters run in different ways.

Suitable ultrafiltration membranes for diafiltration include regenerated cellulose and polyethersulfone-based membranes, such as ULTRACEL and BIOMAX membranes commercially available from MilliporeSigma.

Preferably continuous or constant volume diafiltration is used, where buffer is added at the same rate that filtrate is generated.

The buffer or buffers chosen for the diafiltration step is preferably the buffer desired for the final formulation of the product, e.g., the drug product. In this way, the subsequent virus filtration step is carried out with the final formulation buffer. Those skilled in the art will know what buffers are suitable for the particular product being manufactured. By way of example, for the drug ERBITUX (cetuximab) for I.V. injection, a suitable buffer is 10 mM citric acid monohydrate. For REOPRO (abciximab), a suitable buffer is 70 mM sodium phosphate.

After virus filtration in the final formulation buffer, the product can be concentrated as needed such as by ultrafiltration.

What is claimed is:

1. A method for purifying a sample comprising a biomolecule of interest and impurities, comprising expressing said biomolecule of interest in a bioreactor to form a product sample comprising said biomolecule of interest and impurities; subjecting said product sample to filtration to form a product sample; subjecting said product sample to affinity chromatography to remove impurities from said product sample, and subsequently subjecting said product sample to diafiltration prior to virus filtration.

2. The method of claim 1, wherein said affinity chromatography comprises Protein A affinity ligand.

3. The method of claim 1, further comprising subjecting said concentrated product sample to a virus inactivation step upstream of said diafiltration.

4. The method of claim 3, further comprising subjecting said c product sample to a polishing step downstream of said virus inactivation step and upstream of said diafiltration.

5. The method of claim 4, wherein said polishing step comprising one or more of anion exchange chromatography, cation exchange chromatography, and hydrophobic interaction chromatography.

6. The method of claim 1, wherein the biomolecule is an antibody selected from the group consisting of a recombinant antibody, a recombinant monoclonal antibody, a polyclonal antibody, a humanized antibody and an antibody fragment.

7. The method of claim 1, wherein said biomolecule is a protein.

8. The method of claim 1, wherein said biomolecule is a virus.

9. The method of claim 1, wherein said diafiltration step includes a diafiltration buffer, and wherein said virus filtration includes a virus filtration a virus filtration buffer having substantially the same composition as said diafiltration buffer.

10. A system for purifying a biomolecule of interest, comprising:
a. a bioreactor;
b. a filtration unit downstream of said bioreactor for clarifying the product sample exiting from said bioreactor;
c. at least two affinity chromatography columns configured in series downstream of said filtration unit for receiving the clarified product stream from said filtration unit;
d. a virus inactivation filter positioned downstream of said at least two affinity chromatography columns;
e. one or more anion exchange, cation exchange or hydrophobic interaction exchange chromatography columns positioned downstream of said virus inactivation filter;
f. a diafiltration unit positioned downstream of said one or more anion exchange, cation exchange or hydrophobic interaction exchange chromatography columns; and
g. a virus filtration unit positioned downstream of said diafiltration unit.

11. The system of claim 10, wherein said at least two affinity chromatography columns each comprise Protein A affinity ligand.

12. The system of claim 10, configured to operate in a batch mode.

13. The system of claim 10, configured to operate in a continuous mode.

14. A system for purifying a biomolecule of interest, comprising:
a. a bioreactor;
b. a filtration unit downstream of said bioreactor for clarifying the product sample exiting from said bioreactor;
c. at least two affinity chromatography membrane units configured in series downstream of said filtration unit for receiving the clarified product stream from said filtration unit;
d. a virus inactivation filter positioned downstream of said at least two affinity chromatography membrane units;
e. one or more anion exchange, cation exchange or hydrophobic interaction exchange chromatography membrane unit(s) positioned downstream of said virus inactivation filter;
f. a diafiltration unit positioned downstream of said one or more anion exchange, cation exchange or hydrophobic interaction exchange chromatography membrane unit(s); and
g. a virus filtration unit positioned downstream of said diafiltration unit.

15. The system of claim 14, wherein said at least two affinity chromatography units each comprise Protein A affinity ligand.

16. The system of claim 14, configured to operate in a batch mode.

17. The system of claim 14, configured to operate in a continuous mode.
